(19) United States
(12) Patent Application Publication
(10) Pub. No.: US 2024/0043604 A1
(43) Pub. Date: Feb. 8, 2024

(54) UPGRADED STABILIZED POLYOL COMPOSITION

(71) Applicant: Huntsman International LLC, The Woodlands, TX (US)

(72) Inventor: Leema Joseph, Mumbai (IN)

(21) Appl. No.: 18/266,442

(22) PCT Filed: Nov. 18, 2021
(86) PCT No.: PCT/EP2021/082166

§ 371 Date: Jun. 9, 2023

(30) Foreign Application Priority Data
Nov. 20, 2020 (IN) 202011050656
Jan. 8, 2021 (EP) 211506514

(51) Int. Cl. C08G 18/48; C08J 11/28

(52) U.S. Cl. CPC C08G 18/4833; C08J 11/28; C08J 2375/08

(57) ABSTRACT

The present invention relates to a method for reducing the acid value in a provided polyol to obtain an upgraded stabilized polyol composition, said method comprising following process steps:
Solubilizing ammonia in a distillable alcohol having a boiling point lower than 200° C. with formation of an ammoniated distillable alcohol;
Providing a polyol having a predefined acid value;
Chemically reacting the ammoniated distillable alcohol with the provided polyol;
Removing the distillable alcohol by distillation at a temperature comprised between 120 and 220° C.; and
Obtaining an upgraded stabilized polyol composition having an acid value lower than the predefined acid value of said provided polyol.

The present invention relates to an upgraded stabilized polyol composition.

Typically, polyurethanes consist of polymers composed of a chain of organic units joined by urethane linkages resulting from the reaction between an isocyanate group and an isocyanate-reactive group, preferably a hydroxyl group. Industrially, polyurethane polymers are usually formed by reacting a polyisocyanate with a polyol, wherein both the polyisocyanate and the polyol contain on average two or more functional groups per molecule.

Polyurethanes can be produced in many different forms from very low-density foams to high performance composites and can thus be used in a multitude of applications: flexible foams, rigid foams, footwears, adhesives, coatings, and more generally in elastomer, insulation, building, construction and automotive. More specific examples of applications include flexible high-resilience foam seating, rigid foam insulation panels, electrical potting compounds, high-performance adhesives, surface coatings, packaging, surface sealants and synthetic fibers.

Polyisocyanate (PU) products or polyisocyanurate (PIR) products used in some applications, will have a certain lifetime and, when they should be replaced, they can be disposed of, as wastes. This has a negative impact on the environment.

There is a need to recycle these PU or PIR wastes through cost-efficient processes, while respecting the environment.

It is known that PU or PIR products can be converted into polyols through (chemical) reactions. With this, it is therefore possible to recuperate a part of the waste, such as polyols.

There are several processes available, such as hydrolysis (addition of water), aminolysis (addition of amine), acidolysis (addition of acid), alcoholysis (addition of alcohol) and glycolysis (addition of glycol, e.g. DE 2 238 109, DE 2 557 172, DE 2 711 145 and DE 2 834 431). The skilled person is well aware of all methods suitable for converting PU/PIR products (wastes thereof) into polyols.

In the alcoholysis of PU products, use is made of hydroxyl groups in the form of diols and/or triols to break urethane groups.

Other methods can also consist in dissolving PU waste in glycols (glycolysis) at elevated temperature and precipitating amines by means of hydrogen chloride. In addition, there is also the possibility of dissolving PU waste in diols, precipitating amines by means of halogenated esters of phosphoric acid, removal of the amine salts and reaction with isocyanates (U.S. Pat. No. 4,044,046).

Unfortunately, the polyol recuperated in the end of the extraction process has a relatively high acid value, which is higher than the value of the polyol used as raw material (“fresh polyol”) for forming the initial PU product. The acids present in that polyol waste can react with catalysts, when a foam is prepared. This adversely affects the curing profile, demolding time and/or foam properties, when such polyol waste is used into PU or PIR formulations. Moreover, when polyols with high acid value are used for the preparation of PU products, several terminal-acid groups can form an amide bond in the reaction with polyisocyanates leading to the formation of carbon dioxide. Undesirable bubble formation is therefore observed. This impacts the quality of the final product having inappropriate mechanical properties.

This means that the obtained polyols cannot be used in several fields of applications, as the one mentioned hereinbefore. The user has therefore a minimum of latitude, when using the obtained polyol for preparing PU or PIR end products, with uncertainties about the quality of the polyol used.

There is therefore a need to further process polyol wastes, preferably coming from PU or PIR wastes, in order to be able to recycle these polyol wastes in valuable products in PU and/or PIR applications.

It is an object of the present invention to overcome the aforementioned drawbacks by providing a technical solution for recycling a polyol waste with a simple, cost-efficient and environmentally friendly process.

In this respect, the present invention provides an upgraded stabilized polyol composition obtainable by the following process steps:
Solubilizing ammonia in a distillable alcohol with formation of a solution of aminated distillable alcohol;
Providing a polyol (waste polyol) having a predefined acid value;
Chemically reacting the solution of ammoniated distillable alcohol with the provided polyol (waste polyol); and
Obtaining the upgraded stabilized polyol composition having an acid value lower than the predefined acid value of said provided polyol (waste polyol).

In the context of the invention, we have noticed that polyol waste can be upgraded by the application of the process of the present invention, without the need to use corrosive acids.

It should be understood that “fresh/virgin polyol” having a certain acid value is combined with polyisocyanates in order to achieve PU or PIR products having a certain lifetime. There are several known processes for recuperating the polyol when these products are no longer used. Acidolysis process is one example of these processes. In the end of the process, the polyol waste has an acid value higher than the acid value of the raw material, “fresh/virgin polyol”, used for providing these initial PU or PIR products.

In the present invention, the provided process enables reducing the acid value of that recuperated polyol (waste polyol) in order to provide an upgraded stabilized polyol composition, which has an acid value which is equal to or lower than the acid value of the recuperated polyol (waste polyol). This is particularly advantageous, since the upgraded stabilized polyol composition obtained with the present invention can be directly used, and thereby recycled (preferably, by excluding the use of strong acid and base). The process of the invention is therefore green in that sense and respects the environment. Moreover, an additional technical effect provided by the upgraded stabilized polyol composition of the invention relates to the improvement of the reactivity profile compared to the one of the polyol wastes. Moreover, that reactivity profile of the upgraded stabilized polyol composition can be similar or even enhanced compared to “fresh/virgin polyol”, which is particularly advantageous.

In a further preferred embodiment, we have also noticed that the polyol waste can also be a “fresh polyol”, in order to further reduce its acid value, for specific applications, where low acid values are required. With this, it is also possible to reach an acid value down to 0.03 mg KOH/g, when a “fresh polyol” is used or when a given polyol waste is used. This is particularly advantageous. The present invention enables to provide a process, which is particularly efficient at reducing the acid vale of a given polyol, i.e. “fresh/virgin polyols”, polyol waste, etc. This specific embodiment can be combined with any other embodiment/option referred in the present invention, including any recited (in)dependent claims.

The first step of the process of the invention consists in solubilizing ammonia in a distillable alcohol with formation of a solution of ammoniated distillable alcohol.

Ammonia as referred herein is preferably in gaseous form, which will be solubilized in a distillable alcohol. The solution obtained can be reacted with polyol waste, which has a certain acid value. This is a chemical reaction between the ammonia and the distillable alcohol, preferably ethylene glycol, in order to decrease the acid value of that polyol waste. A simple physical mixing is not enough to reach the effects provided by the invention.

Preferably, that polyol waste comes from PU products or PIR products, in particular from used footwear, used elastomers, used PU or PIR foams and mixtures thereof.

Advantageously, the polyol of the present invention can be used for providing different types of products, such as flexible foams, rigid foams, footwears, adhesives, coatings, but also in elastomer, insulation, building, construction and automotive. More specific examples of applications include flexible high-resilience foam seating, rigid foam insulation panels, electrical potting compounds, high-performance adhesives, surface coatings, packaging, surface sealants and synthetic fibers.

More precisely, the aforementioned processes, which are suitable for converting wastes of PU products or PIR products into polyol wastes can be used in the present invention and are therefore part of it. The skilled person is well aware of all possible processes for recuperating polyols from used PU or PIR products.

The chemical reaction between the ammoniated distillable alcohol and the polyol waste enables reducing the acid value of that polyol waste, which is, in the end of the process, stable enough and upgraded in the sense that it can be directly used in several fields of technology. The user has the freedom to integrate the polyol of the invention directly into different PU or PIR systems. The lifetime of the polyol used as raw material for preparing new PU or PIR products can therefore be extended, thanks to the process of the present invention.

One additional advantage of the process of the present invention is linked to the fact that the process is environmentally friendly, contrarily to known methods known in the art, which makes the invention particularly attractive.

More advantageously, said solution of ammoniated distillable alcohol is added to said polyol waste at an equivalent weight ratio comprised between 0.25 and 3, preferably between 0.25 and 2.5, more preferably between 1 and 2.2. This embodiment enables to further reduce the acidity of the final polyol.

According to a preferred embodiment of the present invention, after reacting said solution of ammoniated distillable alcohol with the provided polyol waste, the distillable alcohol is separated from said solution leading to a further reduction of the acid value of said upgraded stabilized polyol composition.

With this embodiment, the acid value of the polyol of the present invention can go down to 0.08 mg KOH/g, preferably down to 0.03 mg KOH/g.

In an advantageous embodiment of the present invention, the upgraded stabilized polyol composition has a moisture content lower than the moisture content of said polyol waste, in particular after said separation step of said distillable alcohol.

In a particular embodiment, the reaction between said solution of ammoniated distillable alcohol with said provided polyol waste is carried out at a temperature from 70 to 140° C.

Preferably, said reaction between said solution of ammoniated distillable alcohol with said provided polyol waste is carried out at a first temperature comprised between 70 to 95° C., and is followed by an increase of temperature comprised between 95 and 140° C.

More preferably, said solution of ammoniated distillable alcohol comprises between 5 and 25% by weight of ammonia or derivative thereof, preferably between 5 and 20% by weight, based on the total weight of said solution of ammoniated distillable alcohol.

With this feature, it was noticed that a solution is obtained, wherein the ammonia is sufficiently solubilized into the distillable alcohol, which is advantageous, when that solution is mixed with the polyol waste.

According to a particularly advantageous embodiment of the invention, said distillable alcohol is separated from said solution of ammoniated distillable alcohol at a temperature comprised between 120 and 220° C., more preferably between 140° C. and 200° C., for example by distillation, advantageously under vacuum, in order to remove water and said distillable alcohol.

In a preferred embodiment, said distillable alcohol has a boiling point lower than 200° C.

More preferably, said distillable alcohol is selected from the group comprising ethylene glycol, methanol, phenol, ethanol, 1,2 propylene glycol, 1,3 butanediol, 1,4 butanediol.

According to an additional embodiment, said polyol waste comes from a used polyurethane/polyisocyanurate based product, preferably used footwear, used foams, used elastomers or mixtures thereof. The idea is to recuperate the polyol from a starting material which has to be normally disposed of or destroyed. With this, any wastes of PU or PIR products can be used to provide the polyol waste as defined in the present invention. Preferably, that polyol waste comes from PU products or PIR products, in particular from used footwear, used elastomers, used PU or PIR foams and mixtures thereof.

As explained hereinabove, polyols are used as raw materials for providing PU or PIR products. Such “fresh/virgin” polyols have an acid value, which does not adversely affect the properties of the polyols, which can be directly used.

The present invention enables providing a process, which can upgrade and stabilize a polyol waste leading to the formation of a polyol, which can be directly used in new applications with efficiency in terms of the (mechanical) properties obtained.

According to a particular embodiment of the present invention, the polyol waste is polyether-based polyol.

In one embodiment of the present invention, the upgraded stabilized polyol composition is further mixed with other compounds selected from the group comprising surfactants, catalysts, additives, additional polyols and mixtures thereof with formation of a polyol blend, wherein the acid value of the obtained polyol blend remains equal (or close) to the acid value of said upgraded and stabilized polyol composition.

The polyol of the present invention can be mixed with a “fresh/virgin polyol” in order to provide a polyol blend, which can be further mixed with a polyisocyanate.

Regarding the polyol blend, it has been noticed that its OH value can be kept steady as well in such a way that stability (OH value is maintained almost constant over time as well as its viscosity) of that polyol blend can be guaranteed over time.

Other embodiments of the composition of the present invention are mentioned in the annexed claims.

The present invention also relates to polyol, preferably polyether polyol, having an acid value lower than 0.45 mg KOH/g, preferably lower than 0.30 mg KOH/g, more preferably lower than 0.10 mg KOH/g, even more preferably equal to or lower than about 0.08 mg KOH/g, advantageously lower than 0.03 mg KOH/g.

Other embodiments of the polyol of the present invention are mentioned in the annexed claims.

The present invention also concerns the use of a polyol obtained by the process according to the present invention to provide a polyurethane or polyisocyanurate product.

Other embodiments of the use according to the present invention are mentioned in the annexed claims.

Furthermore, the present invention is also about a process for providing an upgraded stabilized polyol composition, preferably having an acid value lower than 0.45 mg KOH/g, which process comprises the following steps:
Solubilizing ammonia in a distillable alcohol with formation of a solution of ammoniated distillable alcohol;
Providing a polyol waste having a predefined acid value;
Chemically reacting the solution of ammoniated distillable alcohol with the provided polyol waste; and
Obtaining the upgraded stabilized polyol composition having an acid value lower than the predefined acid value of said polyol waste.

The present invention hence relates to a method for reducing the acid value in a provided (waste) polyol to obtain an upgraded stabilized polyol composition, said method comprising following process steps:
Solubilizing ammonia in a distillable alcohol having a boiling point lower than 200° C. with formation of an ammoniated distillable alcohol;
Providing a polyol having a predefined acid value;
Chemically reacting the ammoniated distillable alcohol with the provided polyol;
Removing the distillable alcohol by distillation at a temperature comprised between 120 and 220° C.; and
Obtaining an upgraded stabilized polyol composition having an acid value lower than the predefined acid value of said provided polyol.

All above technical features mentioned for the upgraded stabilized polyol composition obtainable by the aforementioned process can also be combined with the features recited for the above process, which provides an upgraded stabilized polyol composition, preferably having an acid value lower than 0.45 mg KOH/g.

Other embodiments of the process according to the present invention are mentioned in the annexed claims.

“Derivatives of ammonia” wording should be understood in the present invention as being amines, which are compounds deriving from ammonia by replacing one, two or three hydrogen atoms by hydrocarbyl groups, and having the general structure R₁NH₂ (primary amines), R₂NH (secondary amines), R₃N (tertiary amines).

Distillable alcohol must be capable of being easily distilled off from the obtained polyol, for instance at atmospheric pressure or, if necessary, under reduced pressure. Preferred ones are aliphatic alcohols of from 1 to 6 carbon atoms, and preferably from 1 to 3 carbon atoms. Examples of suitable alcohols of this kind are N-hexanol, N butanol and t-butanol. Suitable are methanol, ethanol, propanol and isopropanol. The monoalcohols may also be used, if necessary, in the form of commercial products having water contents of about 4% by weight. Phenol may be used instead of the specified monoalcohols. Particularly suitable distillable alcohol can be ethylene glycol.

In the context of the present invention, the expression “solution of aminated distillable alcohol” refers to a solution where ammonia or derivatives thereof are solubilized in a distillable alcohol. When ammonia is used, the expression can be read as follows (in case the distillable alcohol is ethylene glycol): “solution of ammoniated ethylene glycol”. Preferably, ammonia is solubilized in ethylene glycol to give a solution of ammoniated ethylene glycol.

In the invention, the expression “obtainable” can be replaced by “obtained”.

In the invention, the chemical reaction between the solution of ammoniated ethylene glycol and the provided polyol (polyol waste) can be carried out by applying a temperature comprised between 70 and 140° C. More preferably, this can be operated in two steps, where a first temperature comprised between 70 and 95° C. is applied and then a second temperature comprised between 95 and 140° C. is further applied to the mixture comprising the solution of ammoniated distillable alcohol with the polyol waste.

In the end of the process an upgraded stabilized polyol composition can be obtained, which has an acid value lower than the predefined value of the polyol waste.

In a particularly advantageous embodiment of the invention, it has been noticed that the OH value and the viscosity of the upgraded stabilized polyol composition obtained according to the present invention is stable over time, since that OH value remains stable over a long period of time.

The viscosity of the upgraded stabilized polyol composition can range between 26 to 66.6 Pa at 20° C.

The acid value can be analyzed with Metrohm Autotitrator in accordance with the ASTM D 4664 and ASTM D 7253.

The moisture content can be analyzed with Metrohm Autotitrator Karl-Fischer in accordance with ASTM D 203-16 and ASTM D 6304.

In the present invention, the OH value (also referred as OH number or OH content) can be measured according to ASTM D1957 standard and is expressed in mg KOH/g.

OH value can be determined by reacting the hydroxyl groups with an acid anhydride and titrating the acid liberated with potassium hydroxide solution. The unit for OH value is expressed in mg KOH/g polyol. OHv=(56.1 g/mol KOH×polyol functionality×1000)/(molecular weight).

According to an example of the invention, the polyol waste can be reacted with ammonia gas directly solubilized in ethylene glycol, preferably at a temperature of 80 to about 120° C. The solvent ethylene glycol can be distilled out of the reaction mixture under vacuum at a temperature of 120 to 200° C., until the hydroxyl value of the reaction mixture falls down to the hydroxyl value of the polyol waste. With the process of the present invention, the acid value of the polyol waste can be reduced from 0.78 mg KOH/g (polyol waste) down to 0.08 mg KOH/g, preferably down to 0.03 mg KOH/g—upgraded stabilized polyol composition.

Advantageously, the amount of ammonia gas solubilized into the ethylene glycol is 14% (preferably 14.4 wt. %), based on the total weight of the solution of ammoniated ethylene glycol. The ammoniated ethylene glycol can be stored in dark bottle in the refrigerator. The solution of ammoniated ethylene glycol is reacted with the polyol waste at a temperature of 80 to about 120° C. The vacuum distillation of ethylene glycol is carried out at a temperature of 120° C. to 200° C. The polyol waste should chemically react with the solution of ammoniated ethylene glycol.

Since ammonia is preferably used in gaseous form, the moisture content of the upgraded stabilized polyol composition is not increased. Moreover, the distillation step decreases further the moisture content of the upgraded stabilized polyol composition.

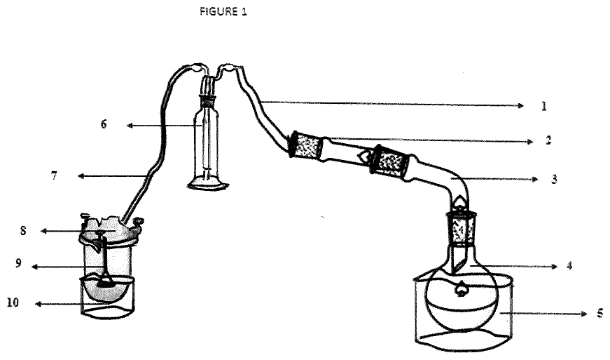

FIG. 1 illustrates the preparation of ammoniated ethylene glycol.

On FIG. 1, the following references are indicated with corresponding objects: 1—PVC tubes; 2—Expansion adapter; 3—Receiving adapter; 4—One-neck flask; 5—Oil batch; 6—Dreschel wash bottles; 7—PVC tubes; 8—Reactor; 9—Funnel; 10—Ice bath.

One aspect of the present invention relates to the solubilization of ammonia into ethylene glycol. The ammoniated ethylene glycol used in the present invention can be prepared by purging ammonia gas into chilled ethylene glycol. An ammonia stream can be generated from a liquor ammonia solution by heating at a temperature of 40° C. to 55° C. in a single neck 500 ml flask. The ammonia stream from the flask is passed through a drying agent to dry the ammonia gas. The ammonia gas dried by passing it through potassium hydroxide pellets is purged into (bubbled through) the chilled ethylene glycol which is stirred vigorously with magnetic stirrer for an hour. The solution was stirred vigorously for 3 to 4 hours or till a 14% of ammonia is incorporated in chilled ethylene glycol. The prepared ammonia in ethylene glycol is stored in the dark amber bottle at 4° C. to avoid sunlight penetration and ammonia loss.

Example 1 indicates a first embodiment of the invention with a preferred description about the process of solubilizing ammonia within ethylene glycol.

### EXAMPLE 1

In this embodiment, ammonia in gaseous form chemically reacts with ethylene glycol leading to the formation of a solution of ammoniated ethylene glycol. Ammonia is solubilized in the ethylene glycol. The ammoniated ethylene glycol solution is prepared by purging ammonia into chilled ethylene glycol. An ammonia stream can be generated from the liquor ammonia solution, by heating it to 40° C. in a single neck 500 ml flask. The ammonia stream from the flask is passed through a drying agent to dry the ammonia gas. The ammonia gas dried by passing it through potassium hydroxide pellets is purged into the chilled ethylene glycol which is stirred vigorously with magnetic stirrer for an hour. The solution was stirred vigorously for 3 to 4 hours or till 14% of ammonia is incorporated in chilled ethylene glycol. The prepared ammonia in ethylene glycol is stored in the dark amber bottle at 4° C. to avoid sunlight penetration and ammonia loss.

In 1 L stirring reaction container, a polyol waste (moisture content of 0.58%) coming from used footwear product having an acid number equal to 0.78 mg KOH/g is provided. In order to improve the miscibility of the polyol waste, the temperature is gradually increased to 80° C. The solution of ammoniated ethylene glycol is added to the polyol waste at a weight ratio of 2:1 at a stirrer speed of 300 rpm and at a temperature of 80° C. The reaction temperature is increased to 120° C. for 1-2 hour. The acid value of the obtained polyol composition is determined: 0.21 mg KOH/g.

In a preferred embodiment, the acid value of the obtained polyol can be further decreased to between 0.08 and 0.21 mg KOH/g by gradually increasing the temperature to 200° C., in order to distill the ethylene glycol out of the obtained polyol. The moisture content of the polyol after distillation is equal to 0.44%.

### EXAMPLE 2—REACTIVITY PROFILE IN FOOTWEAR APPLICATION

In table 1 below, the reactivity profile of three rigid-foam compositions are illustrated, pursuant to PU foam cup test according the ASTM D 7487.

A standard polyol blend is provided by mixing between 80-90 wt. % of a “fresh polyol” having an OH value of 28 mg KOH/g of and a functionality of about 3, with 1.5 wt. % of Niax L6900 silicone surfactant, 2 wt. % of triethylenediamine catalyst, 0.5 wt. % of water and other additives. The mixture is blended at 3000 rpm to form a standard polyol blend, which is further mixed with a polyisocyanate—Suprasec 2444.

Same formulation is obtained (20 wt. % of recycled polyol, 2^{d} column in table 1) by mixing the same components hereinbefore, except that the “fresh polyol” is replaced by a polyol waste with an OH value of 44 mg KOH/g (obtained by acidolysis) with 20 wt. % loading. The polyol blend has a weight ratio “fresh polyol”/polyol waste of 90/10 to 80/20, based on 100 wt. % of the polyol blend.

In the third column in table 1, it can be noticed that a polyol obtained according to the present invention is used. The composition and loadings are the same as the composition with the polyol waste, except that the polyol waste referred hereinbefore is treated by the process of the present invention in order to reduce its acidity.

The properties of the obtained foams are summarized in table 1 hereinbelow.

TABLE 1

| | | | Blend with 20% |
|---|---|---|---|
| | | | of recycled polyol |
| | | Blend with 20% | treated with the |
| | Standard | by wt. of | process of the |
| Reactivity Profile | blend | recycled polyol | present invention |
| Cream time (s) | 19 | 15 | 17 |
| Pinch time (s) | 65 | 100 | 61 |
| Free rise density | 305 | 359.63 | 292 |
| (s) | | | |

Cream time refers to the time required for the reaction mixture to change from the liquid state to a creamy state and starts to foam (expand) subsequently.

Pinch time is the time from beginning the pour until the top of the foam can be pinched without tearing.

Free rise density refers to density measured on foam samples made under atmospheric conditions according to ASTM D 7487.

### COMPARATIVE EXAMPLE 1

Example 1 is repeated, except that the polyol waste with ammoniated ethylene glycol were physically blended (in place of chemically blended). The acid value of the resulted polyol was 0.99 mg KOH/g.

Reference throughout this specification to “one embodiment” or “an embodiment” means that a particular feature, structure or characteristic described in connection with the embodiment is included in at least one embodiment of the present invention. Thus, appearances of the phrases “in one embodiment” or “in an embodiment” in various places throughout this specification are not necessarily all referring to the same embodiment but may. Furthermore, the particular features, structures or characteristics may be combined in any suitable manner, as would be apparent to a person skilled in the art from this disclosure, in one or more embodiments. Furthermore, while some embodiments described herein include some but not other features included in other embodiments, combinations of features of different embodiments are meant to be within the scope of the invention, and form different embodiments, as would be understood by those in the art. For example, in the appended claims, any of the claimed embodiments can be used in any combination.

As used herein, the singular forms “a”, “an”, and “the” include both singular and plural referents unless the context clearly dictates otherwise. By way of example, “an isocyanate group” means one isocyanate group or more than one isocyanate group.

The terms “comprising”, “comprises” and “comprised of” as used herein are synonymous with “including”, “includes” or “containing”, “contains”, and are inclusive or open-ended and do not exclude additional, non-recited members, elements or method steps. It will be appreciated that the terms “comprising”, “comprises” and “comprised of” as used herein comprise the terms “consisting of”, “consists” and “consists of”. This means that, preferably, the aforementioned terms, such as “comprising”, “comprises”, “comprised of”, “containing”, “contains”, “contained of”, can be replaced by “consisting”, “consisting of”, “consists”.

Throughout this application, the term “about” is used to indicate that a value includes the standard deviation of error for the device or method being employed to determine the value.

As used herein, the terms “% by weight”, “wt. %”, “weight percentage”, or “percentage by weight” are used interchangeably.

The recitation of numerical ranges by endpoints includes all integer numbers and, where appropriate, fractions subsumed within that range (e.g. 1 to 5 can include 1, 2, 3, 4 when referring to, for example, a number of elements, and can also include 1.5, 2, 2.75 and 30 3.80, when referring to, for example, measurements). The recitation of end points also includes the end point values themselves (e.g. from 1.0 to 5.0 includes both 1.0 and 5.0). Any numerical range recited herein is intended to include all sub-ranges subsumed therein.

All references cited in the present specification are hereby incorporated by reference in their entirety. In particular, the teachings of all references herein specifically referred to are incorporated by reference.

Unless otherwise defined, all terms used in disclosing the invention, including technical and scientific terms, have the meaning as commonly understood by one of ordinary skill in the art to which this invention belongs. By means of further guidance, term definitions are included to better appreciate the teaching of the present invention.

Throughout this application, different aspects of the invention are defined in more detail. Each aspect so defined may be combined with any other aspect or aspects unless clearly indicated to the contrary. In particular, any feature indicated as being preferred or advantageous may be combined with any other feature or features indicated as being preferred or advantageous. Although the preferred embodiments of the invention have been disclosed for illustrative purpose, those skilled in the art will appreciate that various modifications, additions or substitutions are possible, without departing from the scope and spirit of the invention as disclosed in the accompanying claims.

1. A method for reducing a predefined acid value in a provided polyol to obtain an upgraded stabilized polyol composition, said method comprising:
solubilizing ammonia in a distillable alcohol having a boiling point lower than 200° C. to form an ammoniated distillable alcohol;
chemically reacting the ammoniated distillable alcohol with the provided polyol;
removing the ammoniated distillable alcohol by distillation at a temperature between 120° C. and 220° C.; and
obtaining the upgraded stabilized polyol composition having an acid value lower than the predefined acid value of said provided polyol.

2. The method according to claim 1, wherein said ammoniated distillable alcohol is added to the provided polyol at an equivalent weight ratio between 0.25 and 3.

3. The method according to claim 1, wherein the provided polyol is a waste polyol.

4. The method according to claim 1, wherein the upgraded stabilized polyol composition has a moisture content lower than the moisture content of the provided polyol.

5. The method according to claim 1, wherein the step of removing the distillable alcohol by distillation is performed at a temperature between 140° C. and 200° C. in order to remove water and said distillable alcohol.

6. The method according to claim 1, wherein the chemical reaction between the ammoniated distillable alcohol with said provided polyol is carried out at a temperature from 70° C. to 140° C.

7. The method according to claim 6, wherein said reaction is carried out at a first temperature between 70° C. to 95° C., and is followed by an increase of temperature between 95° C. and 140° C.

8. The method according to claim 1, wherein the ammoniated distillable alcohol comprises between 5% and 25% by weight of ammonia or derivative thereof, based on the total weight of said ammoniated distillable alcohol.

9. The method according to claim 1, wherein said distillable alcohol is selected from the group of ethylene glycol, methanol, phenol, ethanol, 1,2 propylene glycol, 1,3 butanediol, and 1,4 butanediol.

10. The method according to claim 1, wherein the provided polyol is a waste polyol originating from recycling of used polyurethane/polyisocyanurate based products.

11. The method according to claim 3, wherein the waste polyol is a polyether-based polyol.

12. The method according to claim 1, wherein said upgraded stabilized polyol composition is further mixed with another compound selected from the group of a surfactant, a catalyst, an additive and a mixture thereof and wherein the acid value after mixing said compound remains equal or close to the acid value of said upgraded stabilized polyol composition before mixing said compound.

13. The method according to claim 1, wherein said upgraded stabilized polyol composition has an acid value lower than 0.45 mg KOH/g.

14. An upgraded stabilized polyol composition produced according to the method of claim 1.

15. (canceled)

16. The upgraded stabilized polyol composition of claim 14, wherein the provided polyol is a waste polyol.

17. The upgraded stabilized polyol composition of claim 16, wherein the upgraded stabilized composition has an acid value lower than 0.45 mg KOH/g.
